(19) United States
(12) Patent Application Publication
(10) Pub. No.: US 2023/0293111 A1
(43) Pub. Date: Sep. 21, 2023

(54) INFORMATION PROCESSING APPARATUS, INFORMATION PROCESSING METHOD, AND COMPUTER-READABLE MEDIUM

(71) Applicant: Kazuya NIYAGAWA, Kanagawa (JP)

(72) Inventor: Kazuya NIYAGAWA, Kanagawa (JP)

(21) Appl. No.: 18/117,474

(22) Filed: Mar. 6, 2023

(30) Foreign Application Priority Data
Mar. 16, 2022 (JP) 2022-041812

(51) Int. Cl. A61B 5/00

(52) U.S. Cl. CPC A61B 5/721

(57) ABSTRACT

An information processing apparatus includes a first acquisition unit, a separation unit, a first identification unit, and a second identification unit. The first acquisition unit is configured to acquire measurement data being time-series data obtained by measuring a biological signal by a measurement apparatus. The separation unit is configured to separate the measurement data acquired by the first acquisition unit into a plurality of signal components by a multivariate analysis. The first identification unit is configured to identify, from among the plurality of signal components, a signal component including an unwanted component other than a biological signal being a measurement target. The second identification unit is configured to identify, using, as reference data, the signal component identified as including the unwanted component by the first identification unit, a signal component including an unwanted component again from among the plurality of signal components.

### CROSS-REFERENCE TO RELATED APPLICATIONS

The present application claims priority under 35 U.S.C. § 119 to Japanese Patent Application No. 2022-041812, filed on Mar. 16, 2022. The contents of which are incorporated herein by reference in their entirety.

### BACKGROUND OF THE INVENTION

### 1. Field of the Invention

The present invention relates to an information processing apparatus, an information processing method, and a computer-readable medium.

### 2. Description of the Related Art

In electroencephalography (EEG) and magnetoencephalography (MEG) for measuring brain activity, various kinds of noise and biological artifacts originated from living things are mixed in acquired measurement data, in addition to a biological signal. The biological artifacts originated from blinks, motion of a heart, and the like may have higher signal intensity than a biological signal that is a measurement target, and, in a signal analysis for the electroencephalography and the magnetoencephalography, there is a need to remove the biological artifacts as described above. As a method of removing the biological artifacts as described above, it is already known that a method using an independent component analysis (ICA) for separating the measurement data into independent signal components, identifying biological artifacts from the signal components, and removing the biological artifacts from the measurement data is effective.

However, as a conventional method of removing the biological artifacts (in particular, artifacts due to heartbeat) using the ICA, in a method of manually identifying components that seem to be artifacts from among the separated independent components, it may be possible to select artifact components that seem to correspond to all of heartbeat components, but a selection result may vary because the selection depends on abilities and experiences of a person who performs the identification; therefore, accuracy is not constant and a large amount of time and effort are needed for the operation. Furthermore, in a method of automatically identifying components that seem to be artifacts from among the separated independent components, using a predetermined algorithm, there is a need to use data of electro-cardiogram (ECG) or magneto-cardiogram (MGC) that is separately measured as reference data in order to identify components that seem to correspond to all of the heartbeat components with high accuracy. If the reference data as described above is not used, although it may be possible to automatically identify main heartbeat components, if removing performance is reduced and heartbeat components are present over a plurality of independent components or mixed with different signals for example, it is difficult to identify the heartbeat components with high accuracy.

As a technique for detecting an unwanted component in a signal, using the ICA as described above, a configuration that includes a component extracting means for extracting a desired component from a plurality of signal waveforms based on a detected biological signal by performing a principal component analysis or the ICA, a sorting means for sorting a plurality of extraction results obtained by the component extracting means in order from the highest periodicity and displaying the sorted components, and a noise component selecting means for receiving a selection of one of the extraction results obtained by the component extracting means as a noise component has been disclosed (for example, Japanese Unexamined Patent Application Publication No. 2019-154879).

Furthermore, in order to perform a process of removing an unwanted signal from biological signal data, as a technique for removing an unwanted signal component from measurement data, a technique for obtaining correlation between a reference signal that is separately acquired from a reference signal acquisition unit and a biological signal, and removing a noise signal from the biological signal data has been disclosed (for example, Japanese Patent No. 4631510).

However, in the conventional technologies, when the reference data that is separately measured is not used, identification performance of the unwanted component is reduced, which is a problem.

### SUMMARY OF THE INVENTION

According to an aspect of the present invention, an information processing apparatus includes a first acquisition unit, a separation unit, a first identification unit, and a second identification unit. The first acquisition unit is configured to acquire measurement data being time-series data obtained by measuring a biological signal by a measurement apparatus. The separation unit is configured to separate the measurement data acquired by the first acquisition unit into a plurality of signal components by a multivariate analysis. The first identification unit is configured to identify, from among the plurality of signal components, a signal component including an unwanted component other than a biological signal being a measurement target. The second identification unit is configured to identify, using, as reference data, the signal component identified as including the unwanted component by the first identification unit, a signal component including an unwanted component again from among the plurality of signal components.

### BRIEF DESCRIPTION OF THE DRAWINGS

FIG. 1 is a schematic configuration diagram of a biological signal measurement system according to a first embodiment;

FIG. 2 is a diagram illustrating an example of a hardware configuration of an information processing apparatus according to the first embodiment;

FIG. 3 is a diagram illustrating an example of a functional block configuration of the information processing apparatus according to the first embodiment;

FIG. 4 is a diagram illustrating an example of waveforms of signal components that are separated by an independent component analysis;

FIG. 5 is a flowchart illustrating an example of the flow of an artifact identification and removing process performed the biological signal measurement system according to the first embodiment;

FIG. 6 is a diagram illustrating an example of waveforms of signal components before and after removing of an unwanted component;

FIG. 7 is a diagram illustrating an example of a functional block configuration of an information processing apparatus according to a second embodiment; and

FIG. 8 is a flowchart illustrating an example of the flow of an artifact identification and removing process performed by a biological signal measurement system according to the second embodiment.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

Embodiments of an information processing apparatus, an information processing method, and a computer-readable medium according to the present invention will be described in detail below with reference to the drawings. The present invention is not limited by the embodiments below, and components in the embodiments below include one that can easily be thought of by a person skilled in the art, one that is practically identical, one that is what is called an equivalent, and the like. Furthermore, various omission, replacement, modifications, and combinations of the components may be made without departing from the gist of the embodiments described below.

An embodiment has an object to provide an information processing apparatus, an information processing method, and a computer-readable medium capable of preventing a reduction in identification performance of an unwanted component in measurement data even if reference data that is separately measured is not used.

### First Embodiment

### Overview of Biological Signal Measurement System

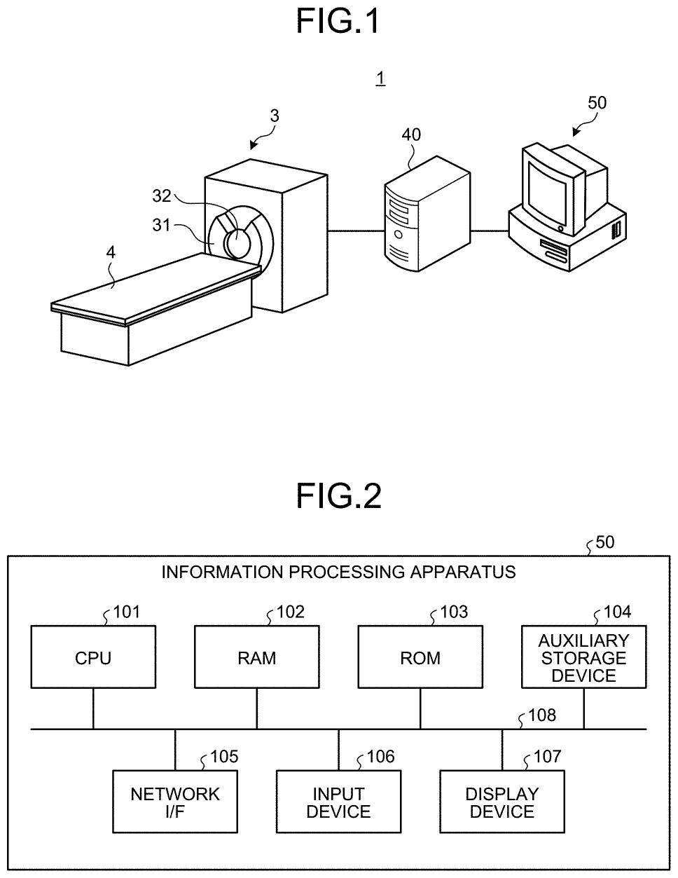

FIG. 1 is a schematic configuration diagram of a biological signal measurement system according to a first embodiment. An overview of a biological signal measurement system 1 according to the present embodiment will be described below with reference to FIG. 1.

A biological signal measurement system 1 is an information processing system that measures and acquires a plurality of kinds of biological signals (for example, magneto-encephalography (MEG) data, electro-encephalography (EEG) data, and the like) of a subject, and removes an unwanted component due to a biological artifact (hereinafter, may be simply referred to as an artifact) from the measured measurement data. Meanwhile, the biological signal to be measured is not limited to the MEG data and the EEG data.

Conventionally, if a biological artifact component, in particular, an artifact component due to heartbeat, is to be removed with high accuracy, using an independent component analysis or the like, there is a need to use certain data (for example, electro-cardiogram (ECG) data or magneto-cardiogram (MGS) data) that is separately measured as a reference, and, if the reference data is not used, removing performance is reduced and the artifact component is not completely removed from the measurement data. In the present embodiment, operation of using, as the reference data, a signal component that is identified as including an obvious artifact component without using the separately measured reference data, performing a process of identifying a signal component that finally includes an artifact component, and removing the artifact component will be described.

As illustrated in FIG. 1, the biological signal measurement system 1 includes a measurement apparatus 3 that measures one or more kinds of biological signals of a subject, a server 40 that accumulates measurement data of the one or more kinds of biological signals that are measured by the measurement apparatus 3, and an information processing apparatus (brain activity determination apparatus) that analyzes the one or more kinds of measurement data that are recorded in the server 40. Meanwhile, in FIG. 1, the server 40 and an information processing apparatus 50 are illustrated as separate apparatuses; however, for example, at least a part of functions of the server 40 may be incorporated in the information processing apparatus 50. Furthermore, in FIG. 1, the information processing apparatus 50 is illustrated as a single information processing apparatus, but embodiments are not limited to this example, and an information processing system that includes a plurality of information processing apparatuses may be applicable.

In the example illustrated in FIG. 1, a subject (to-be-measured person) lies down on a measurement table 4 with face up while electrodes (or sensors) for electroencephalography are mounted on the head, and a head portion is inserted in a hollow 32 of a dewar 31 of the measurement apparatus 3. The dewar 31 is a holding container in an extremely low temperature environment using liquid helium, and a large number of magnetic sensors for magnetoencephalography are arranged inside the hollow 32 of the dewar 31. The measurement apparatus 3 collects electroencephalography data from the electrodes and magnetoencephalography data from the magnetic sensors through measurement, and outputs measurement data or the like that includes the collected electroencephalography data and the collected magnetoencephalography data to the server 40. In this case, the measurement data is time-series data obtained from each of the magnetic sensors and each of the electrodes. The electroencephalography data is a signal that represents electrical activity of a nerve cell (ion charge flow that occurs in dendrites of a neuron at the time of synaptic transmission) as a voltage value between the electrodes. The magnetoencephalography data is a signal that represents minute magnetic field variation that occurs due to electrical activity of a brain. The brain's magnetic field is detected by high-sensitive superconducting quantum interference device (SQUID) sensors. The electroencephalography data and the magnetoencephalography data are one example of a “biological signal”. The measurement data that is output to the server 40 is read, displayed, and analyzed by the information processing apparatus 50. In general, the dewar 31 in which the magnetic sensors are incorporated and the measurement table 4 are arranged in a magnetic shielding room, but illustration of the magnetic shielding room is omitted in FIG. 1 for the sake of convenience.

The information processing apparatus 50 is an apparatus that analyzes the measurement data that includes the magnetoencephalography data obtained from the plurality of magnetic sensors and the electroencephalography data obtained from the plurality of electrodes.

### Hardware Configuration of Information Processing Apparatus

FIG. 2 is a diagram illustrating an example of a hardware configuration of the information processing apparatus according to the first embodiment. The hardware configuration of the information processing apparatus 50 according to the present embodiment will be described below with reference to FIG. 2.

As illustrated in FIG. 2, the information processing apparatus 50 includes a central processing unit (CPU) 101, a random access memory (RAM) 102, a read only memory (ROM) 103, an auxiliary storage device 104, a network interface (I/F) 105, an input device 106, and a display device 107, all of which are connected to one another via a bus 108.

The CPU 101 is an arithmetic device that controls entire operation of the information processing apparatus 50 and performs various kinds of information processing. The CPU 101 executes a program that is stored in the ROM 103 or the auxiliary storage device 104 and controls an artifact identification and removing process (to be described later).

The RAM 102 is a volatile storage device that is used as a work area of the CPU 101 and that stores therein main control parameters and information. The ROM 103 is a non-volatile storage device that stores therein a basic input-output program or the like. For example, it may be possible to store the program as described above in the ROM 103.

The auxiliary storage device 104 is a non-volatile storage device, such as a hard disk drive (HDD) or a solid state drive (SSD). The auxiliary storage device 104 stores therein, for example, a program for controlling the operation of the information processing apparatus 50, various kinds of data and files that are needed for the operation of the information processing apparatus 50, and the like.

The network I/F 105 is a communication interface for performing communication with an apparatus, such as the server 40, on a network. The network I/F 105 is implemented by, for example, a network interface card (NIC) or the like that is compliant with transmission control protocol/Internet protocol (TCP/IP).

The input device 106 is an input function of a touch panel, a user interface, such as a keyboard, a mouse, or an operation button, or the like. The display device 107 is a display device that displays various kinds of information. The display device 107 is implemented by, for example, a display function of a touch panel, a liquid crystal display (LCD), an organic electro-luminescence (EL), or the like.

Meanwhile, the hardware configuration of the information processing apparatus 50 illustrated in FIG. 2 is one example, and a different device may be added. Further, the information processing apparatus 50 illustrated in FIG. 2 has the hardware configuration based on the assumption that the information processing apparatus 50 is a personal computer (PC) for example, but embodiments are not limited to this example, and a mobile terminal, such as a tablet, may be adopted. In this case, it is sufficient that the network I/F 105 is a communication interface with a wireless communication function.

### Functional Block Configuration and Operation of Information Processing Apparatus

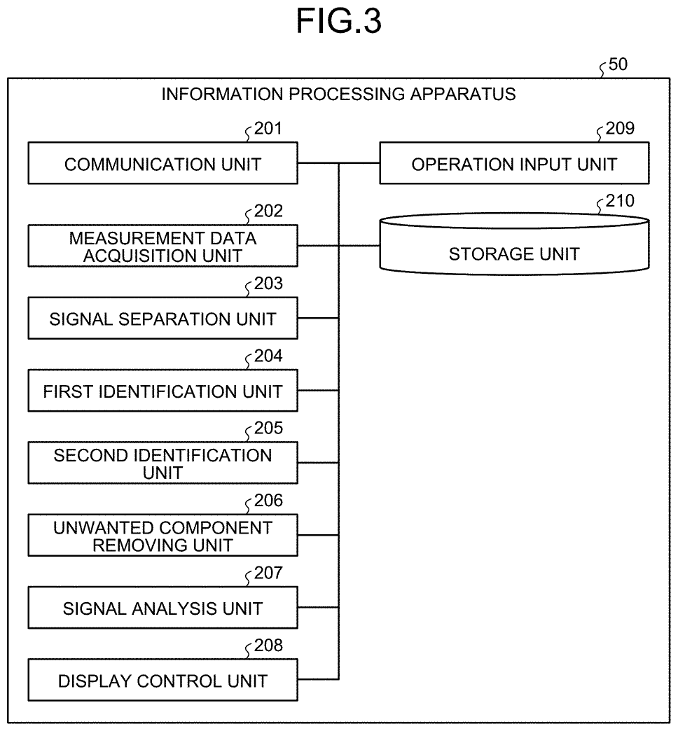

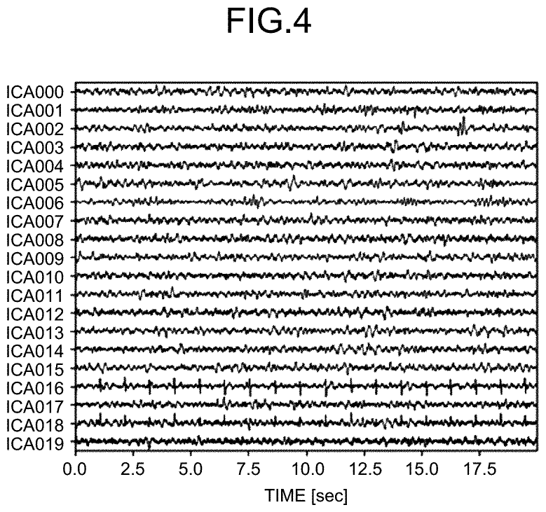

FIG. 3 is a diagram illustrating an example of a functional block configuration of the information processing apparatus according to the first embodiment. FIG. 4 is a diagram illustrating an example of waveforms of signal components that are separated by an independent component analysis. The functional block configuration and the operation of the information processing apparatus 50 according to the present embodiment will be described below with reference to FIG. 3 and FIG. 4.

As illustrated in FIG. 3, the information processing apparatus 50 includes a communication unit 201, a measurement data acquisition unit 202 (first acquisition unit), a signal separation unit 203 (separation unit), a first identification unit 204, a second identification unit 205, an unwanted component removing unit 206 (removing unit), a signal analysis unit 207 (analysis unit), a display control unit 208, an operation input unit 209, and a storage unit 210.

The communication unit 201 is a functional unit that performs data communication with the measurement apparatus 3, the server 40, or the like. For example, the communication unit 201 receives, from the server 40, measurement data that is obtained by measuring a biological signal by the measurement apparatus 3, and stores the measurement data in the storage unit 210. Meanwhile, the communication unit 201 may directly receive the measurement data from the measurement apparatus 3. The communication unit 201 is implemented by the network I/F 105 illustrated in FIG. 2.

The measurement data acquisition unit 202 is a functional unit that acquires the measurement data on the biological signal that is accumulated in the storage unit 210. Meanwhile, the measurement data acquisition unit 202 may directly acquire the measurement data from the measurement apparatus 3 or the server 40 via the communication unit 201.

The signal separation unit 203 is a functional unit that separates the measurement data that is acquired by the measurement data acquisition unit 202 into a plurality of signal components by a multivariate analysis. As the multivariate analysis for separation into the plurality of signal components, for example, an independent component analysis, a principal component analysis (PCA), a nonnegative matrix factorization (NMF), or the like may be adopted. Further, as one example of an algorithm for the independent component analysis, picard or the like may be adopted.

The first identification unit 204 is a functional unit that identifies a single component that includes an obvious artifact component from among all of the signal components that are separated by the signal separation unit 203. Here, the artifact component is an unwanted component other than the biological signal that is a measurement target (for example, the magnetoencephalography data and the electroencephalography data). Specifically, the first identification unit 204 adopts, for example, a kurtosis as an index, divides each of the signal components into epochs each corresponding to a predetermined time (for example, one second), and calculates a kurtosis for each of the epochs. In this case, it is sufficient to determine the predetermined time in accordance with a type of a target artifact component. Here, it is assumed that the Fisher's kurtosis is used as the kurtosis, and the kurtosis has a value that is obtained by dividing a fourth-order central moment by the square of variance and then subtracting three from the quotient. Further, if absolute values of the kurtosis of a predetermined percent (for example, 30%) or more of all of the epochs of the signal component exceed a predetermined threshold (for example, 2), the first identification unit 204 identifies that the signal component includes the artifact component. Meanwhile, the index for identifying the obvious artifact component is not limited to the kurtosis, but, for example, Shannon entropy, a degree of distortion, or the like may be used. Further, as the method of identifying the obvious artifact component, it may be possible to adopt a method of allowing a user to manually selecting a signal component including the artifact component. Furthermore, if a plurality of signal components that are identified as including the artifact components by the first identification unit 204 are present, it may be possible to select a signal component that meets a more preferable condition (for example, the signal component for which the number of epochs that meet the condition for the kurtosis is the largest) among the conditions as described above, or it may be possible to randomly select a signal component from among the plurality of signal components.

The second identification unit 205 is a functional unit that adopts the signal component that is identified as including the artifact component by the first identification unit 204 as pseudo reference data, and identifies a signal component including the artifact component again, using an identification algorithm using the pseudo reference data with respect to all of the signal components that are separated by the signal separation unit 203. Here, cross trial phase statistics (CTPS) is used as the identification algorithm, for example. CTPS is a method of segmentation into trials each corresponding to one second before and after a peak (R peak in electro-cardiogram) of a waveform that is obtained from the reference data, calculation of a “cross-trial phase distribution” at each time between the trials, and calculation of a degree of deviation of a distribution from a uniform distribution. Meanwhile, as the identification algorithm, it may be possible to use an algorithm for performing identification by obtaining correlation between the reference data and the signal component.

The identification process performed by the first identification unit 204 and the second identification unit 205 will be described below with reference to FIG. 4. Waveforms illustrated in FIG. 4 are one example of waveforms of all of signal components that are separated from the measurement data by the signal separation unit 203. If the first identification unit 204 performs the identification process on each of the signal components, using the kurtosis as an index, it is identified that the signal component “ICA016” illustrated in FIG. 4 includes an artifact component. In contrast, for example, the signal component “ICA018” seems to include a heartbeat component that is an artifact component; however, because an absolute value of the kurtosis is small, the signal component “ICA018” is not identified as including the artifact component in the identification process performed by the first identification unit 204. Further, the second identification unit 205 performs an identification process on a signal component including the artifact component through CTPS, using the signal component “ICA016” that is identified as the first identification unit 204 as the pseudo reference data, so that the signal component “ICA018” is identified as including the artifact component.

The unwanted component removing unit 206 is a functional unit that removes, from the measurement data, the artifact component identified by the second identification unit 205.

The signal analysis unit 207 is a functional unit that performs an analysis process, such as dipole estimation, on the measurement data from which the artifact component that is an unwanted component has been removed by the unwanted component removing unit 206.

The display control unit 208 is a functional unit that controls display operation of the display device 107. For example, the display control unit 208 causes the display device 107 to display the measurement data before and after removing in order to check whether the artifact component has been removed, or causes the display device 107 to display an analysis result obtained by the signal analysis unit 207.

The operation input unit 209 is a functional unit that receives input of operation. The operation input unit 209 is implemented by the input device 106 illustrated in FIG. 2.

The storage unit 210 is a functional unit that stores therein the measurement data or the like that is received by the communication unit 201. The storage unit 210 is implemented by the RAM 102 or the auxiliary storage device 104 illustrated in FIG. 2.

The measurement data acquisition unit 202, the signal separation unit 203, the first identification unit 204, the second identification unit 205, the unwanted component removing unit 206, the signal analysis unit 207, and the display control unit 208 as described above are implemented by causing the CPU 101 to load a program that is stored in the ROM 103 or the like onto the RAM 102 and execute the loaded program. Meanwhile, a part or all of the measurement data acquisition unit 202, the signal separation unit 203, the first identification unit 204, the second identification unit 205, the unwanted component removing unit 206, the signal analysis unit 207 and the display control unit 208 may be implemented by a hardware circuit, such as an application specific integrated circuit (ASIC) or a field-programmable gate array (FPGA), instead of a program that is software.

Meanwhile, each of the functional units illustrated in FIG. 3 is a functionally conceptual, and need not always be configured in the same manner. For example, a plurality of functional units that are illustrated as independent functional units in FIG. 3 may be configured as a signal functional unit. In contrast, a function included in a single functional unit in FIG. 3 may be divided into a plurality of functions, and may be configured as a plurality of functional units.

### Flow of Artifact Identification and Removing Process

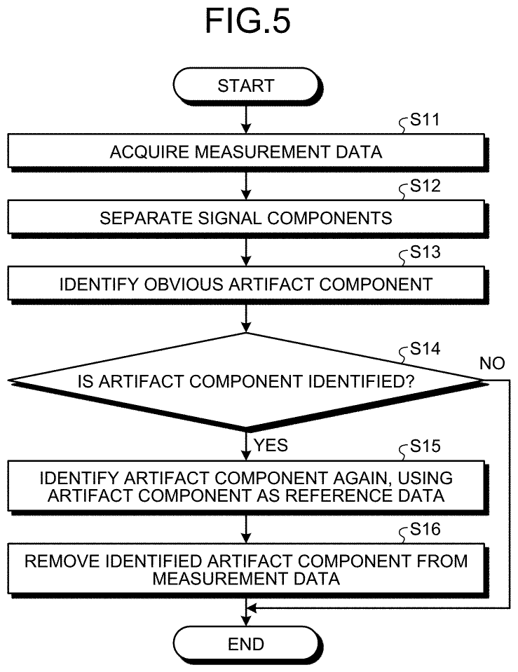

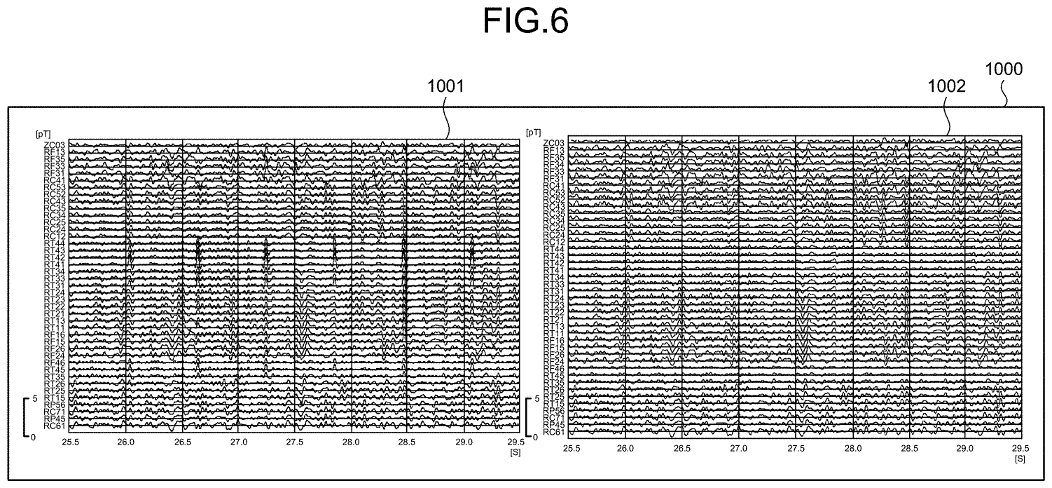

FIG. 5 is a flowchart illustrating an example of the flow of the artifact identification and removing process in the biological signal measurement system according to the first embodiment. FIG. 6 is a diagram illustrating an example of waveforms of signal components before and after removing of an unwanted component. The flow of the artifact identification and removing process performed by the information processing apparatus 50 of the biological signal measurement system 1 according to the present embodiment will be described below with reference to FIG. 5 and FIG. 6.

### Step S11

The measurement data acquisition unit 202 of the information processing apparatus 50 acquires measurement data of a biological signal that is accumulated in the storage unit 210. Meanwhile, the measurement data acquisition unit 202 may directly acquire the measurement data from the measurement apparatus 3 or the server 40 via the communication unit 201. Then, the process goes to Step S12.

### Step S12

The signal separation unit 203 of the information processing apparatus 50 separates the measurement data that is acquired by the measurement data acquisition unit 202 into a plurality of signal components by a multivariate analysis. Then, the process goes to Step S13.

### Step S13

The first identification unit 204 of the information processing apparatus 50 identifies a signal component that includes an obvious artifact component from among all of the signal components that are separated by the signal separation unit 203. Specifically, the first identification unit 204 adopts, for example, a kurtosis as an index, divides each of the signal components into epochs each corresponding to a predetermined time (for example, one second), and calculates the kurtosis for each of the epochs. Then, if absolute values of the kurtosis of a predetermined percent (for example, 30%) or more of all of the epochs of the signal component exceed a predetermined threshold (for example, 2), the first identification unit 204 identifies that the signal component includes the artifact component. Then, the process goes to Step S14.

### Step S14

If the first identification unit 204 identifies the signal component including the artifact component (YES at Step S14), the process goes to Step S15. If the signal component is not identified (NO at Step S14), any process is not performed, and the artifact identification and removing process is terminated.

### Step S15

The second identification unit 205 of the information processing apparatus 50 adopts the signal component that is identified as including the artifact component by the first identification unit 204 as pseudo reference data, and identifies a signal component including the artifact component again, using an identification algorithm using the pseudo reference data with respect to all of the signal components that are separated by the signal separation unit 203. Then, the process goes to Step S16.

### Step S16

The unwanted component removing unit 206 of the information processing apparatus 50 removes the artifact component that is identified by the second identification unit 205 from the measurement data. Then, the artifact identification and removing process is terminated.

Thereafter, the signal analysis unit 207 performs an analysis process, such as dipole estimation, on the measurement data from which the artifact component that is an unwanted component has been removed by the unwanted component removing unit 206. Then, the display control unit 208 causes the display device 107 to display a signal waveform display screen 1000 as illustrated in FIG. 6 for indicating the measurement data before and after removing in order to check whether the artifact component has been removed, or causes the display device 107 to display an analysis result obtained by the signal analysis unit 207. The signal waveform display screen 1000 illustrated in FIG. 6 includes a pre-removing waveform display region 1001 for displaying waveforms of the respective signal components before removing of the artifact component and a post-removing waveform display region 1002 for displaying waveforms of the respective signal components after removing of the artifact component.

As described above, in the information processing apparatus 50 of the biological signal measurement system 1 according to the present embodiment, the measurement data acquisition unit 202 acquires the measurement data that is time-series data obtained by measuring a biological signal by the measurement apparatus 3, the signal separation unit 203 separates the measurement data acquired by the measurement data acquisition unit 202 into a plurality of signal components by a multivariate analysis, the first identification unit 204 identifies, from among the plurality of signal components, a signal component that includes an unwanted component other than the biological signal that is a measurement target, and the second identification unit 205 adopts the signal component that is identified as including the unwanted component by the first identification unit 204 as reference data and identifies a signal component including an unwanted component again from among the plurality of signal components. With this configuration, even if reference data that is separately measured is not used, it is possible to prevent reduction in identification performance of the unwanted component in the measurement data.

Furthermore, in the information processing apparatus 50 of the biological signal measurement system 1 according to the present embodiment, the unwanted component removing unit 206 removes, from the measurement data, the unwanted component that is identified by the second identification unit 205. With this configuration, even if the reference data that is separately measured is not used, it is possible to achieve the same removing performance of the unwanted component from the measurement data as the removing performance that is achieved with use of the reference data.

Moreover, in the information processing apparatus 50 of the biological signal measurement system 1 according to the present embodiment, the display control unit 208 causes the display device 107 to display a waveform of the measurement data from which an unwanted component has not yet been removed by the unwanted component removing unit 206 and a waveform of the measurement data from which the unwanted component has been removed. With this configuration, it is possible to check whether the artifact component is effectively removed.

### Second Embodiment

A biological signal measurement system according to a second embodiment will be described below mainly in terms of a difference from the biological signal measurement system 1 according to the first embodiment. In the first embodiment, the operation has been described in which the identification process is finally performed, using the signal component that is identified as including the obvious artifact component as the reference data without using the reference data that is separately measured. In the present embodiment, operation will be described in which, if reference data that is separately measured is present, the identification process is performed using the separately measured reference data. Meanwhile, an overall configuration of the biological signal measurement system according to the present embodiment and a hardware configuration of an information processing apparatus are the same as those described in the first embodiment.

### Functional Configuration and Operation of Information Processing Apparatus

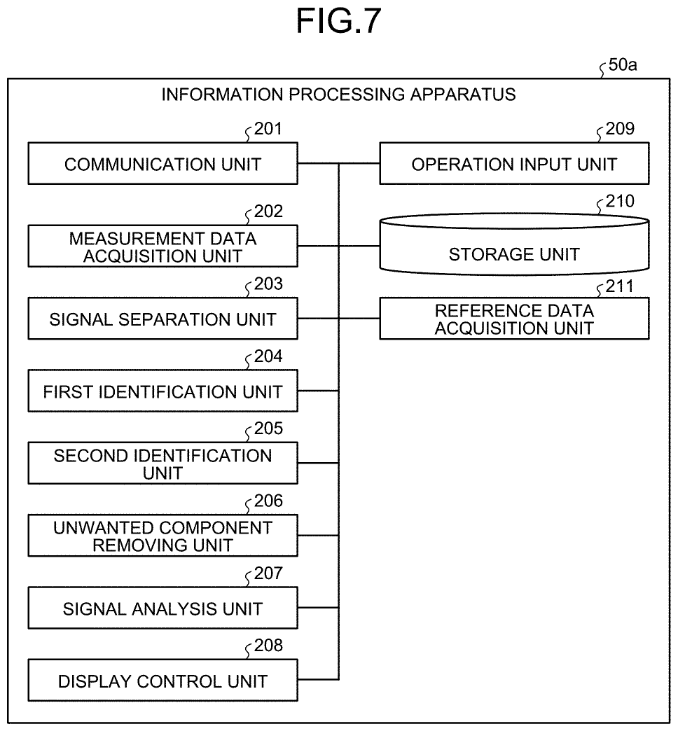

FIG. 7 is a diagram illustrating an example of a functional block configuration of an information processing apparatus according to the second embodiment. A functional block configuration and operation of an information processing apparatus 50a according to the present embodiment will be described below with reference to FIG. 7.

As illustrated in FIG. 7, the information processing apparatus 50a includes the communication unit 201, the measurement data acquisition unit 202 (first acquisition unit), the signal separation unit 203 (separation unit), the first identification unit 204, the second identification unit 205, the unwanted component removing unit 206 (removing unit), the signal analysis unit 207 (analysis unit), the display control unit 208, the operation input unit 209, the storage unit 210, and a reference data acquisition unit 211 (second acquisition unit).

The reference data acquisition unit 211 is a functional unit that acquires, as reference data, data of electro-cardiogram or magneto-cardiogram that is measured by an apparatus different from the measurement apparatus 3 (for example, a magnetoencephalography, a electroencephalography, or the like). For example, the reference data acquisition unit 211 acquires data of electro-cardiogram, magneto-cardiogram, or the like that is measured by an external measurement apparatus (that is, different from the measurement apparatus 3) via the communication unit 201. Meanwhile, if the storage unit 210 stores therein data of electro-cardiogram, magneto-cardiogram, or the like that is separately measured, the reference data acquisition unit 211 may acquire the data as the reference data from the storage unit 210.

The signal separation unit 203, if the reference data that needs to be acquired by the reference data acquisition unit 211 is not present, separates the measurement data that is acquired by the measurement data acquisition unit 202 into a plurality of signal components by a multivariate analysis.

The second identification unit 205, if the reference data that needs to be acquired by the reference data acquisition unit 211 is not present, adopts the signal component that is identified as including the artifact component by the first identification unit 204 as pseudo reference data, and identifies a signal component including the artifact component again using an identification algorithm using the pseudo reference data with respect to all of the signal components that are separated by the signal separation unit 203. Further, if the reference data that needs to be acquired by the reference data acquisition unit 211 is present, the second identification unit 205 identifies the artifact component of the measurement data by an identification algorithm, such as CTPS, using the reference data that is acquired by the reference data acquisition unit 211.

Meanwhile, operation of the functional units other than the reference data acquisition unit 211, the signal separation unit 203, and the second identification unit 205 among the functional units of the information processing apparatus 50a is the same as the first embodiment as described above.

The measurement data acquisition unit 202, the signal separation unit 203, the first identification unit 204, the second identification unit 205, the unwanted component removing unit 206, the signal analysis unit 207, the display control unit 208, and the reference data acquisition unit 211 as described above are implemented by causing the CPU 101 to load a program that is stored in the ROM 103 or the like onto the RAM 102 and execute the program. Meanwhile, a part or all of the measurement data acquisition unit 202, the signal separation unit 203, the first identification unit 204, the second identification unit 205, the unwanted component removing unit 206, the signal analysis unit 207, the display control unit 208, and the reference data acquisition unit 211 may be implemented by a hardware circuit, such as an ASIC or an FPGA, instead of a program that is software.

Meanwhile, each of the functional units illustrated in FIG. 7 is a functionally conceptual, and need not always be configured in the same manner. For example, a plurality of functional units that are illustrated as independent functional units in FIG. 7 may be configured as a signal functional unit. In contrast, a function included in a single functional unit in FIG. 7 may be divided into a plurality of functions, and may be configured as a plurality of functional units.

### Flow of Artifact Identification and Removing Process

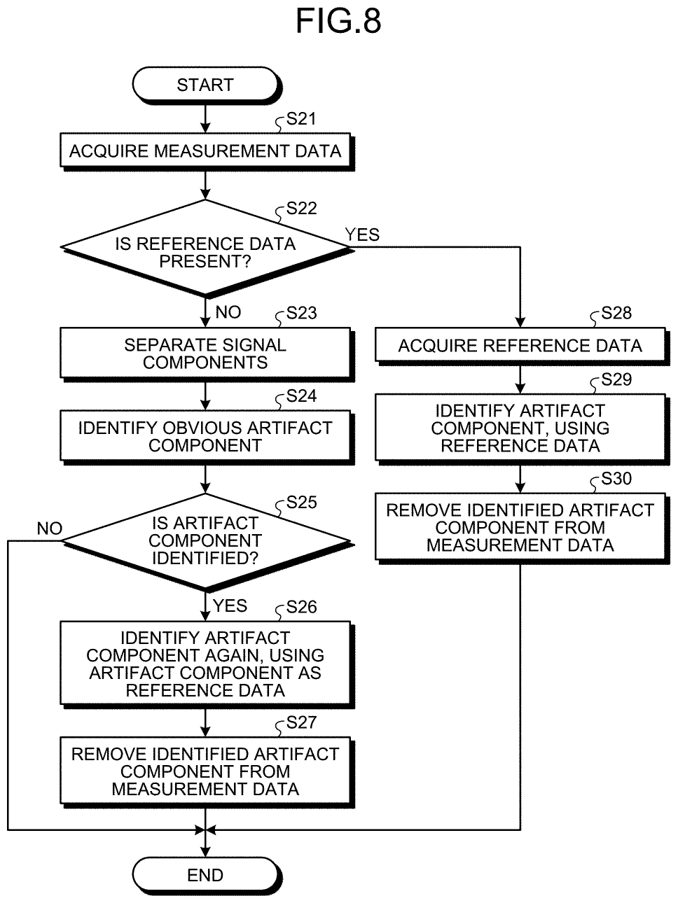

FIG. 8 is a flowchart illustrating an example of the flow of the artifact identification and removing process in the biological signal measurement system according to the second embodiment. The flow of the artifact identification and removing process performed by the information processing apparatus 50a of the biological signal measurement system according to the present embodiment will be described below with reference to FIG. 8.

### Step S21

The measurement data acquisition unit 202 of the information processing apparatus 50a acquires measurement data of a biological signal that is accumulated in the storage unit 210. Meanwhile, the measurement data acquisition unit 202 may directly acquire the measurement data from the measurement apparatus 3 or the server 40 via the communication unit 201. Then, the process goes to Step S22.

### Step S22

If the reference data that needs to be acquired by the reference data acquisition unit 211 is not present (NO at Step S22), the process goes to Step S23. If the reference data is present (YES at Step S22), the process goes to Step S28.

Steps S23 to S27

Processes from Steps S23 to S27 are the same as the processes from Steps S12 to S16 illustrated in FIG. 5 as described above. Then, the artifact identification and removing process is terminated.

### Step S28

The reference data acquisition unit 211 of the information processing apparatus 50a acquires, as the reference data, data of electro-cardiogram, magneto-cardiogram, or the like that is separately measured. For example, the reference data acquisition unit 211 acquires the data of electro-cardiogram, magneto-cardiogram, or the like that is measured by an external measurement apparatus via the communication unit 201. Meanwhile, if the storage unit 210 stores therein the data of electro-cardiogram, magneto-cardiogram, or the like that is separately measured, the reference data acquisition unit 211 may acquire the data as the reference data from the storage unit 210. Then, the process goes to Step S29.

### Step S29

The second identification unit 205 of the information processing apparatus 50a identifies the artifact component of the measurement data by the identification algorithm, such as CTPS, using the reference data that is acquired by the reference data acquisition unit 211. Then, the process goes to Step S30.

### Step S30

The unwanted component removing unit 206 of the information processing apparatus 50a removes the artifact component that is identified by the second identification unit 205 from the measurement data. Then, the artifact identification and removing process is terminated.

As described above, in the information processing apparatus 50a of the biological signal measurement system according to the present embodiment, the reference data acquisition unit 211 acquires the reference data other than the biological signal that is measured by the measurement apparatus 3. Further, if the reference data that is acquired by the reference data acquisition unit 211 is present, the second identification unit 205 identifies the unwanted component from the measurement data, using the reference data. In contrast, if the reference data that is acquired by the reference data acquisition unit 211 is not present, the first identification unit 204 identifies, from among the plurality of signal components, a signal component that includes an unwanted component other than the biological signal that is a measurement target. With this configuration, in addition to achieving the same effects as those of the first embodiment as described above, it is possible to select an optimal method of identifying the unwanted component for each of a case where the separately measured reference data is present and a case where the separately measured reference data is not present, so that it is possible to realize an identification process with high accuracy.

Meanwhile, in the embodiments as described above, if at least one of the functional units of the information processing apparatuses 50 and 50a is implemented by execution of a program, the program is provided by being incorporated in a ROM or the like in advance. Further, the program that is executed by the information processing apparatuses 50 and 50a according to the embodiments as described above may be provided by being recorded in a computer readable recording medium, such as a compact disc (CD)-ROM, a flexible disk (FD), a compact disk recordable (CD-R), or a digital versatile disk, in a computer-installable or computer-executable file format.

Furthermore, the program that is executed by the information processing apparatuses 50 and 50a of the embodiments as described above may be provided by being stored in a computer that is connected to a network, such as the Internet, and by being downloaded via the network. Moreover, the program that is executed by the information processing apparatuses 50 and 50a of the embodiments as described above may be provided or distributed via a network, such as the Internet. Furthermore, the program that is executed by the information processing apparatuses 50 and 50a of the embodiments as described above has a module structure that includes at least any of the functional units as described above, and as an actual hardware, each of the functional units as described above is loaded and generated on a main storage device by causing the CPU to read the program from the ROM or the like.

According to an embodiment, even if reference data that is separately measured is not used, it is possible to prevent reduction in identification performance of an unwanted component of measurement data.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

What is claimed is:

1. An information processing apparatus comprising:
a first acquisition unit configured to acquire measurement data being time-series data obtained by measuring a biological signal by a measurement apparatus;
a separation unit configured to separate the measurement data acquired by the first acquisition unit into a plurality of signal components by a multivariate analysis;
a first identification unit configured to identify, from among the plurality of signal components, a signal component including an unwanted component other than a biological signal being a measurement target; and
a second identification unit configured to identify, using, as reference data, the signal component identified as including the unwanted component by the first identification unit, a signal component including an unwanted component again from among the plurality of signal components.

2. The information processing apparatus according to claim 1, further comprising a removing unit configured to remove, from the measurement data, the unwanted component identified by the second identification unit.

3. The information processing apparatus according to claim 2, further comprising a display control unit configured to display, on a display device, a waveform of the measurement data from which the unwanted component has not yet been removed by the removing unit, and a waveform of the measurement data from which the unwanted component has been removed by the removing unit.

4. The information processing apparatus according to claim 1, further comprising a second acquisition unit configured to acquire reference data other than the biological signal measured by the measurement apparatus, wherein
the first identification unit is configured to identify the signal component including the unwanted component other than the biological signal being the measurement target, from among the plurality of signal components, in a case where the reference data other than the biological signal, the reference data being acquired by the second acquisition unit, is not present, and
the second identification unit is configured to identify the unwanted component from the measurement data, using the reference data other than the biological signal, in a case where the reference data other than the biological signal, the reference data being acquired by the second acquisition unit, is present.

5. The information processing apparatus according to claim 1, wherein the multivariate analysis is one of an independent component analysis and a principal component analysis.

6. The information processing apparatus according to claim 2, further comprising an analysis unit configured to preform a predetermined analysis process on the measurement data from which the unwanted component is removed by the removing unit.

7. An information processing method comprising:
acquiring measurement data being time-series data obtained by measuring a biological signal by a measurement apparatus;
separating the acquired measurement data into a plurality of signal components by a multivariate analysis;
identifying, from among the plurality of signal components, a signal component including an unwanted component other than a biological signal being a measurement target;
identifying, using, as reference data, the signal component identified as including the unwanted component, a signal component including an unwanted component again from among the plurality of signal components.

8. A non-transitory computer-readable medium including programmed instructions that cause a computer to execute:
acquiring measurement data being time-series data obtained by measuring a biological signal by a measurement apparatus;
separating the acquired measurement data into a plurality of signal components by a multivariate analysis;
identifying, from among the plurality of signal components, a signal component including an unwanted component other than a biological signal being a measurement target;
identifying, using, as reference data, the signal component identified as including the unwanted component, a signal component including an unwanted component again from among the plurality of signal components.
